(19) United States
(12) Patent Application Publication
(10) Pub. No.: US 2024/0058538 A1
(43) Pub. Date: Feb. 22, 2024

(54) DEVICES AND METHODS FOR DELIVERING RECONSTITUTED MEDICAMENTS

(71) Applicant: kaleo, Inc., Richmond, VA (US)

(72) Inventors: Glen L. KELLEY, Glen Allen, VA (US); Paul F. MEYERS, Fishers, IN (US); Michael J. ROE, Powhatan, VA (US)

(73) Assignee: kaleo, Inc., Richmond, VA (US)

(21) Appl. No.: 18/227,702

(22) Filed: Jul. 28, 2023

Related U.S. Application Data
(60) Provisional application No. 63/398,410, filed on Aug. 16, 2022.

(51) Int. Cl. A61M 5/20

(52) U.S. Cl. CPC A61M 5/2066; A61M 2005/206; A61M 2005/2073; A61M 2207/10

(57) ABSTRACT

A method of producing a medicament container assembly that contains a dry medicament and a solvent, and having reduced gas content within the dry medicament is described herein. The medicament container assembly includes a container body, a first elastomeric member, a second elastomeric member, and a distal seal. A first elastomeric member, a second elastomeric member, and a portion of the container body collectively define a first volume. The second elastomeric member and a distal end portion of the container body define a second volume. The dry medicament is conveyed into the second volume via a distal end opening of the container body. The solvent is conveyed into the first volume via a proximal end opening. The first elastomeric member is then inserted into the container body via the proximal end opening to seal the solvent within the first volume.

### CROSS-REFERENCE TO RELATED APPLICATIONS

This application claims benefit of priority to U.S. Provisional Application No. 63/398,410, entitled “Devices and Methods for Delivering Reconstituted Medicaments,” filed Aug. 16, 2022, which is incorporated herein by reference in its entirety.

### BACKGROUND

The embodiments described herein relate generally to medicament containers and medicament delivery devices, and more particularly to a medicament delivery device for reconstituting a medicament and delivering the reconstituted medicament into a patient.

Exposure to certain substances, such as, for example, peanuts, shellfish, bee venom, certain drugs, toxins, and the like, can cause allergic reactions in some individuals. Such allergic reactions can, at times, lead to anaphylactic shock, which can cause a sharp drop in blood pressure, hives, and/or severe airway constriction. Similarly, certain medical conditions, such as diabetes, can result in rapid changes or reductions in blood glucose levels, resulting in an emergency condition requiring immediate treatment. As another example, exposure to toxins or nerve agents (e.g., sarin or other organophosphorus compounds) requires immediate treatment. Accordingly, responding rapidly to mitigate the effects from such conditions or exposures is important to prevent injury and/or death. For example, in certain situations, an injection of epinephrine (i.e., adrenaline) can provide substantial and/or complete relief from the allergic reaction. In other situations, for example, an injection of an antidote to a toxin, such as atropine, pralidoxime and midazolam, can greatly reduce and/or eliminate the harm potentially caused by the exposure. Similarly, an injection of glucagon can reduce and/or eliminate the harm potentially caused by reduced blood glucose levels in individuals experiencing a hypoglycemic emergency.

Because emergency medical facilities are not always available when an individual is suffering from such emergencies or medical conditions, some individuals carry an auto-injector, a rescue inhaler, or the like to rapidly self-administer a medicament in response to such emergency medical conditions. Some known auto-injectors include a vial containing a liquid medicament and a spring loaded needle to automatically penetrate the user's skin and inject the medicament. The storage of certain medicaments in a liquid form, however, can result in a shorter shelf life and/or an unstable medicament. Accordingly, some known auto-injectors include a vial containing a first medicament that is separated from a second medicament. Such auto-injectors are often referred to as “wet/dry” auto-injectors, because one substance is often a liquid (e.g., water or another solvent) and the other substance can be substantially solid or dry (e.g., lyophilized glucagon powder). Lyophilization is also known as “freeze drying.” In use, the first medicament and the second medicament must be mixed prior to injection.

Some known wet/dry injectors, however, require multiple steps to reconstitute the dry medicament and are therefore not suited for rapid deployment. For example, some known wet/dry injector require that the user manually actuate a mixing mechanism prior to injection (e.g., by twisting a portion of the device to complete the mixing step). Such configurations can, however, result in incomplete mixing and/or an injection occurring without mixing. As another example, the operation of some known wet/dry delivery systems includes manually inserting the needle into the skin prior to activation and subsequent medicament delivery. The operation of such configurations may also include separately attaching a needle to prepare the device for injection, resulting in a delay in delivery of the medicament. Moreover, such configurations can be complicated, making them difficult for a user to operate during an emergency or by an individual without medical training.

Some known wet/dry injectors require that a user manually vent and/or purge a portion of air included in the medicament container (e.g., mixed with or a part of the glucagon powder). In some instances, such known injectors are generally oriented in a predetermined manner (e.g., with the needle end facing upward) during the mixing process and/or prior to injection to facilitate the venting process (also referred to as “priming”). The use of such injectors, however, may not be practical in certain emergency situations, such as, for example, in a combat setting.

Some known wet/dry injectors employ a single mechanism to automatically mix and inject the medicaments contained therein. Although simpler to use, such known devices may not properly deliver the correct dose. For example, because the mixing operation and the injection operation are actuated together in such configurations, the medicament can sometimes be injected prior to the completion of the mixing operation and/or prior to the injector being properly positioned for the injection operation. Additionally, if excessive air is present in the reconstituted medicament, the accuracy of the delivered dose may be compromised.

Additionally, although many known auto-injectors are carried by individuals in ordinary walks of life, such known devices may not be suitable for storage, transportation, or use in extreme periods for long durations of time. For example, some known auto-injectors may not be able to withstand high altitudes, temperature extremes, or shock (e.g., from dropping the device) that accompany use in military applications.

Thus, a need exists for improved medicament delivery devices to improve the procedures for mixing a medicament and accurately delivering the desired dose of reconstituted medicament. Specifically, a need exists for an improved medicament container assembly that has a reduced amount of air within the dry medicament. A need also exists for an auto-injector that can withstand the harsh environment that can exist in military applications.

### SUMMARY

Medicament delivery devices and methods for producing medicament delivery devices for reconstituting a medicament and delivering the medicament are described herein. In some embodiments, a method of producing a medicament container assembly that contains a dry medicament and a solvent, and having reduced gas content within the dry medicament is described herein. The medicament container assembly includes a container body, a first elastomeric member, a second elastomeric member, and a distal seal. The first elastomeric member is disposed within a proximal end portion of the container body. The second elastomeric member is disposed within the container body distally from the first elastomeric member. The first elastomeric member, the second elastomeric member, and a portion of the container body collectively define a first volume. The second elastomeric member and a distal end portion of the container body define a second volume. The method includes conveying the dry medicament into the second volume via a distal end opening of the container body. The second elastomeric member is within the container body at a first distance from a distal end portion of the container body during the conveying. After the conveying, the second elastomeric member is moved distally within the container body to a second distance from the distal end portion of the container body, the second distance being less than the first distance. The distal end opening is sealed by installing the distal seal about the distal end opening. The solvent is conveyed into the first volume via a proximal end opening. The first elastomeric member is then inserted into the container body via the proximal end opening to seal the solvent within the first volume.

In some embodiments, the dry medicament is a lyophilized medicament and the conveying of the dry medicament includes first conveying a liquid medicament into the second volume, followed by lyophilizing the liquid medicament to produce the lyophilized medicament. In other embodiments, the conveying the dry medicament into the second volume is performed via a dry fill process.

In some embodiments, the moving the second elastomeric member causes a gas within the dry medicament to be expelled from the second volume via the distal end opening. In some embodiments, the moving the second elastomeric member causes a size of the second volume to be decreased by at least fifty percent.

In some embodiments, the distal end portion of the container body includes a neck and the second volume includes a neck portion. The method further includes inserting, after the conveying the dry medicament and before the moving the second elastomeric member, a portion of a bleed fixture into the neck portion of the second volume via the distal end opening. The bleed fixture is configured to allow a gas within the dry medicament to be expelled from the second volume via the distal end opening while limiting the movement of the dry medicament into the neck portion of the second volume.

In some embodiments, an apparatus includes a medicament container assembly that contains a dry medicament and a solvent where the gas contained within the dry medicament is reduced.

In some embodiments, an apparatus includes a housing, a medicament container assembly, and a carrier. The housing has an inner wall that defines a medicament cavity. The medicament container assembly is at least partially disposed within the medicament cavity and contains a dose of a medicament. The carrier is configured to move within the medicament cavity from a first carrier position to a second carrier position in response to an actuation force. The carrier includes a side wall that includes a retention portion and defines a coupling volume. The carrier is coupled to a needle having a proximal tip that extends into the coupling volume. The retention portion of the carrier is configured to retain a distal end portion of the medicament container in a first container position. The distal end portion of the medicament container is fluidically isolated from the proximal tip of the needle when the medicament container is in the first container position. The distal end portion of the medicament container is configured to move to a second container position in response to the actuation force, such that the distal end portion of the medicament container is fluidically coupled to the proximal tip of the needle. The inner wall of the housing and the side wall of the carrier are configured to limit deformation of the retention portion to prevent the medicament container from moving to the second container position when the carrier is in the first carrier position.

### BRIEF DESCRIPTION OF THE DRAWINGS

FIGS. 1A-1F are cross-sectional views of a medicament container assembly in various stages of production using a method according to an embodiment.

FIG. 1G is flow chart of a method of producing a medicament container assembly, according to an embodiment.

FIG. 2 is a front perspective view of a medicament delivery device according to an embodiment in a first (storage) configuration.

FIG. 3 is a side view of the medicament delivery device shown in FIG. 2 in the first (storage) configuration.

FIG. 4 is a front view of the medicament delivery device shown in FIG. 2 with the outer casing removed.

FIG. 5 is a top perspective view of a housing of the medicament delivery device shown in FIG. 2.

FIGS. 6 and 7 are a front perspective view (FIG. 6) and a bottom perspective view (FIG. 7) of a top cap of the medicament delivery device shown in FIG. 2.

FIG. 8 is a cross-sectional view of the medicament delivery device shown in FIG. 2 taken along line X-X in FIG. 3, the medicament delivery device being in the first (storage) configuration.

FIG. 9 is a front perspective view of a cover (also referred to as a case) of the medicament delivery device shown in FIG. 2.

FIG. 10 is a top perspective view of a safety lock of the medicament delivery device shown in FIG. 2.

FIG. 11 is a top perspective view of a base (which functions as an actuator) of the medicament delivery device shown in FIG. 2.

FIGS. 12 and 13 are cross-sectional views of a distal portion (FIG. 12) and a proximal portion (FIG. 13) of a carrier and medicament container assembly the medicament delivery device shown in FIG. 2.

FIG. 14 is a cross-sectional view of the medicament delivery device shown in FIG. 2 taken along line X-X in FIG. 3, the medicament delivery device being in a second configuration (with the cover and safety lock removed).

FIG. 15 is a cross-sectional view of the medicament delivery device shown in FIG. 2 taken along line X-X in FIG. 3, the medicament delivery device being in a third configuration (after being activated).

FIG. 16 is a cross-sectional view of the medicament delivery device shown in FIG. 2 taken along line X-X in FIG. 3, the medicament delivery device being in a fourth configuration (with the bypass flow being initiated).

FIG. 17 is a cross-sectional view of the medicament delivery device shown in FIG. 2 taken along line X-X in FIG. 3, the medicament delivery device being in a fifth configuration (with the bypass flow being completed).

FIG. 18 is a cross-sectional view of the medicament delivery device shown in FIG. 2 taken along line X-X in FIG. 3, the medicament delivery device being in a sixth configuration (with the injection being completed).

FIG. 19 is a cross-sectional view of the medicament delivery device shown in FIG. 2 taken along line X-X in FIG. 3, the medicament delivery device being in a seventh configuration (with the retraction completed).

### DETAILED DESCRIPTION

Medicament container assemblies and methods for producing medicament container assemblies that contain a dry medicament and a solvent are described herein. The medicament container assemblies can be included within any of the medicament delivery devices described herein, and have a reduced amount of gas (e.g., air) in the volume that contains the dry medicament. By reducing the amount of gas contained within the dry medicament, the medicament container assemblies can be used within devices (as described herein) that do not require a separate priming step to allow the excess gas to be bled from the reconstituted drug prior to delivery, while still maintaining the desired dose accuracy. This arrangement can facilitate the medicament delivery devices and drug products described herein, which can be actuated by a single operation to initiate reconstitution, needle insertion, and drug delivery. Additionally, the medicament container assemblies described herein include features (e.g., shaped bypass channels) and are configured to be manipulated (e.g., via the desired force exerted on the plungers) such that the dry medicament is reconstituted rapidly (quasi-instantaneously) by the flow of the solvent into the dry medicament without requiring additional steps (e.g., shaking, inverting the device, or the like).

Accordingly, the medicament delivery devices described herein are suitable for use in a variety of emergency situations where more complex, multi-step procedures are not desirable. Such situations can include, for example, drug delivery in a decentralized setting, in a battlefield setting, in a mass delivery setting (vaccinations, delivery of antidote for nerve agents, or the like).

Additionally, medicament delivery devices that are well suited to be stored for long durations (up to 2 years, up to 5 years, up to 10 years) in rugged environments are described herein. The medicament delivery devices described herein are configured with multiple different levels of safety to prevent premature actuation or inadvertent piercing of the medicament container (e.g., due to being dropped, shaken, stored in environments with changing ambient conditions, etc.).

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

As used herein, the term “medicament” includes any constituent of a therapeutic substance. A medicament can include such constituents regardless of their state of matter (e.g., solid, liquid or gas). Moreover, a medicament can include the multiple constituents that can be included in a therapeutic substance in a mixed state, in an unmixed state and/or in a partially mixed state. A medicament can include both the active constituents and inert constituents of a therapeutic substance. Accordingly, as used herein, a medicament can include non-active constituents such as, water, colorant or the like.

The term “about” when used in connection with a referenced numeric indication means the referenced numeric indication plus or minus up to 10 percent of that referenced numeric indication. For example, “about 100” means from 90 to 110.

As used herein, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device. As another example, the distal end portion of a medical injector is the end from which a needle or delivery member extends during the delivery event.

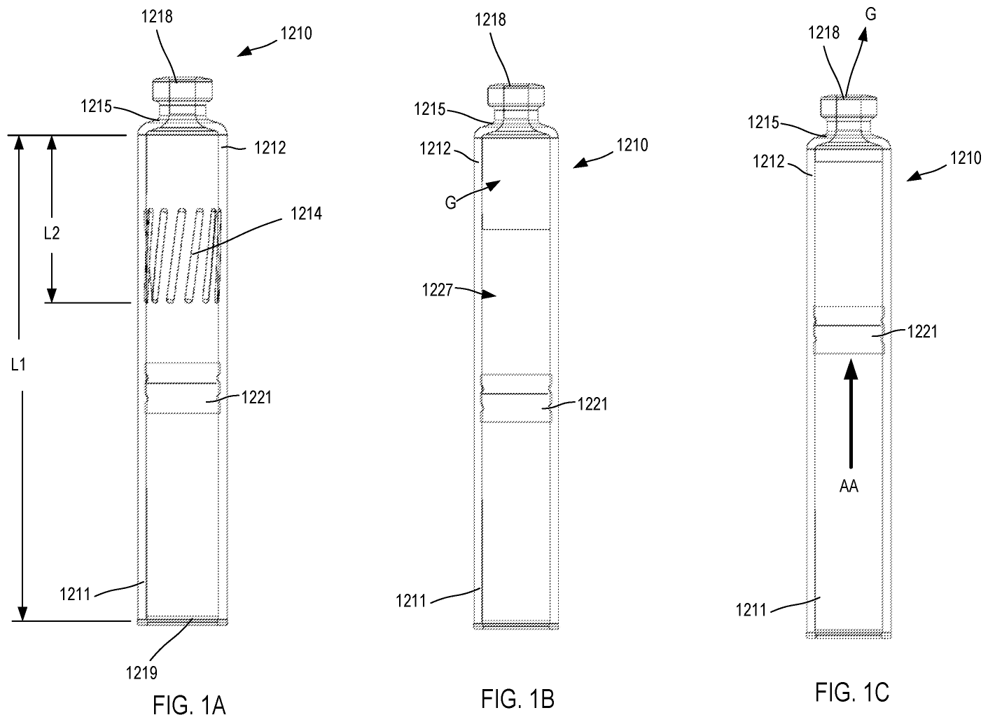

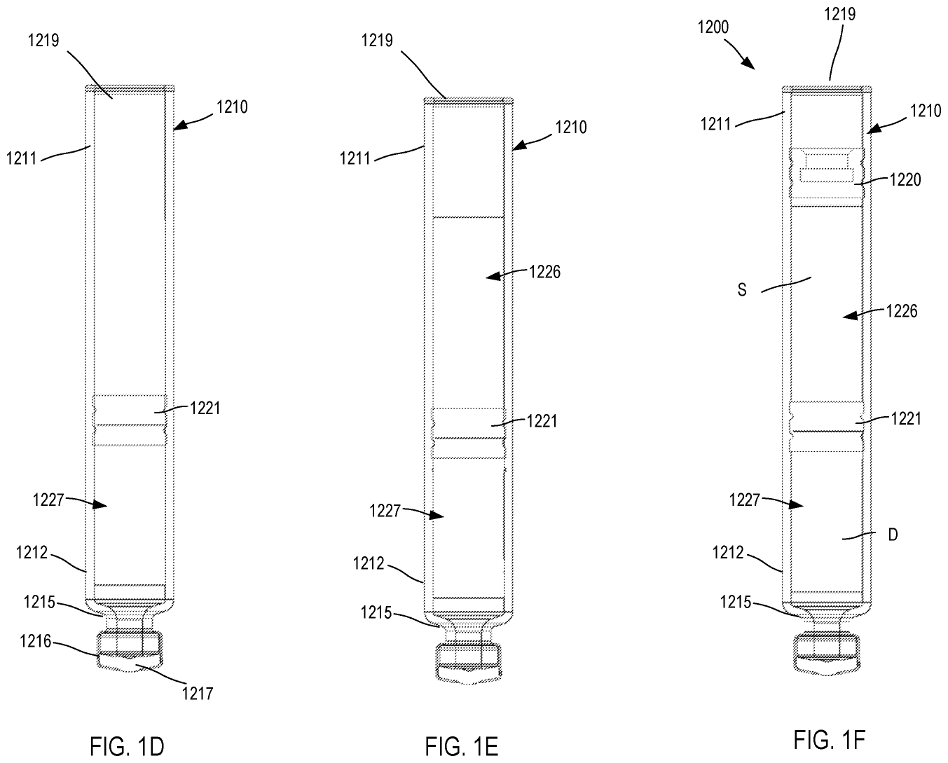

FIGS. 1A-1F show a medicament container assembly 1200 in various stages of production (i.e., of the fill and finish operation) according to an embodiment, with FIG. 1F showing the completed medicament container assembly. FIG. 1G is a flow chart of a method of producing the medicament container assembly according to an embodiment. Upon completion the medicament container assembly will contain a solvent S and a dry medicament D with a reduced amount of gas therein. Referring to FIG. 1F, the completed medicament container assembly 1200 includes a container body 1210 having a proximal end portion 1211, a distal end portion 1212, and defining a proximal end opening 1219 and a distal end opening 1218.

The distal end portion 1212 of the container body 1210 includes a neck 1215 and a distal cap 1216 including a seal member 1217. The distal end portion 1212 is configured to be at least partially disposed within a retention portion 1284 of the carrier 1260, as described below. The distal cap 1216 can be, for example, a crimp seal or cap disposed about the distal end portion 1212 of the container body 1210. The seal member 1217 can be any suitable member, such as, for example, a septum, a valve, a frangible seal, and/or the like. In this manner, the seal member 1217 is configured to engage a surface of the container body 1210 and an inner surface of the distal cap 1216 to define a fluidic seal.

The proximal end opening 1219 of the container body 1210 allows the inner volume 1213 to receive a first elastomeric member 1220 and a second elastomeric member 1221. The first elastomeric member 1220 and the second elastomeric member 1221 are placed within the container body 1210 during the fill/finish process described herein to define a solvent volume 1226 and a dry medicament volume 1227. Said another way, the solvent volume 1226 is a volume disposed within the container body 1210 defined between the first elastomeric member 1220 and the second elastomeric member 1221. The dry medicament volume 1227 is a volume disposed within container body 1210 defined between the second elastomeric member 1220 and the seal member 1217 disposed at the distal end portion 1212 of the container body 1210. As shown in FIG. 1F, the solvent volume 1226 and the dry medicament volume 1227 are defined by the positions of the first elastomeric member 1220 and the second elastomeric member 1221 relative to and/or within the container body 1210. In some embodiments, the solvent volume 1226 can contain a medicament solvent S, such as, for example, an aqueous solvent. In some embodiments, the solvent S includes an alcohol or a miscible organic solvent. In some embodiments, the solvent S includes a surfactant. In some embodiments, the dry medicament volume 1227 can contain a dry medicament D, which can be produced within the container body 1210 via a lyophilization process or alternatively, can be dry filled. The dry medicament D can include any of the formulations and/or compositions described herein.

The first elastomeric member 1220 and the second elastomeric member 1221 can be of any design or formulation suitable for contact with the medicament (e.g., the solvent contained in the solvent volume 1226 and/or a lyophilized or dry filled medicament contained in the dry medicament volume 1227). For example, the elastomeric members 1220 and 1221 can be formulated to minimize any reduction in the efficacy of the medicament that may result from contact (either direct or indirect) between the elastomeric members 1220 and 1221 and the medicament. In some embodiments, the elastomeric members 1220 and 1221 can be made from and/or can include butyl rubber, such as chlorobutyl rubber, bromobutyl rubber, and/or the like. In some embodiments, the first elastomeric member 1220 and the second elastomeric member 1221 can be formulated to minimize any leaching or out-gassing of compositions that may have an undesired effect on the medicament. In other embodiments, the elastomeric members 1220 and 1221 can be formulated to maintain its chemical stability, flexibility and/or sealing properties when in contact (either direct or indirect) with the medicament over a long period of time (e.g., for up to six months, one year, two years, five years or longer).

As shown in FIG. 1A, the container body also includes one or more bypass channels 1214. The bypass channels 1214 are configured to facilitate the mixing and reconstitution of a dry medicament D contained within the container body 1210, as described in further detail herein. In particular, the bypass 1214 is configured to place the dry medicament volume 1227 and the solvent volume 1226 in fluid communication with each other. The container body 1210 has a length L1 and the proximal-most portion of the bypass channels 1214 is located a distance L2 from the distal end portion 1212 of the container body. As described below, including the bypass channels as far distal as possible can allow for the size of the dry medicament volume 1227 to be as small as possible, thus allowing for reduced amount of excess gas therein. Although shown as being a series of channels, in other embodiments, the bypass 1214 can be a singular channel bypass. Although the bypass 1214 is shown as being an internal bypass, in other embodiments, the bypass 1214 can be an external bypass (e.g., a portion that protrudes from the outer surface of the container body. Said another way, in some embodiments a bypass can be configured such that the outer diameter of the container body 1210 is substantially constant.

Referring to FIG. 1G, a method 10 of producing a medicament container assembly (e.g., the medicament container assembly 1200) includes conveying the dry medicament into the dry medicament volume (also referred to as the second volume via a distal end opening (e.g., the distal end opening 1218) of the container body, at 12. The second elastomeric member is within the container body at a first distance from a distal end portion of the container body during the conveying. In some embodiments, the conveying can be performed by initially filling the second volume with a liquid containing the desired drug. For example, in some embodiments, the proximal end portion 1211 of the container body 1210 can be inserted into a tray such that the container body 1210 is secured relative to other components of the system (not shown). With the container body 1210 secured, a component of a filling system (not shown) can deliver a solution containing the desired dosage of medicament via the distal end opening 1218. The method can then optionally include lyophilizing the liquid medicament to produce the lyophilized medicament within the dry medicament volume 1227. As shown in FIG. 1B, as a part of this process the liquid medicament will only partially fill the dry medicament volume 1227 to allow ample volume for air to escape during the lyophilization process. As such, the dry medicament volume 1227 will also include a gas G.

Although the dry medicament is described as being conveyed into the container body via a lyophilization process, in other embodiments, the dry medicament can be dry filled into the container body. For example, in some embodiments, the dry medicament is produced by any of a spray drying process or a micronizing process, and is conveyed into the container body in its dry form.

To reduce the amount of gas in the assembly, the method further includes moving the second elastomeric member distally within the container body to a second distance from the distal end portion of the container body, at 13. This is shown in FIG. 1C by the arrow AA, which shows the second elastomeric member 1221 being moved distally. As shown, the second distance is less than the first distance, so excess gas G is pushed out of the distal end opening 1218 and the overall size of dry medicament volume 1227 is decreased. In some embodiments, the size of the dry medicament volume can be reduced to a value of less than about 1 mL (e.g., between 0.1 mL and 1 mL; between 0.5 mL and 1 mL; and between 0.8 mL and 1.2 mL). In some embodiments, the size of the dry medicament volume can be reduced to be a size of about 50 percent (i.e., half) of its initial value, about 25 percent of its initial value, or about 10 percent of its initial value.

To accommodate the reduction in the size of the dry medicament volume, the bypass channels are positioned closer to the distal end portion 1212 of the container body 1210. In this manner, the second elastomeric member 1221 can be maintained proximally of the proximal-most portion of the bypass channel (to seal the dry medicament D within the dry medicament volume 1227). In some embodiments, the ratio of the length L2 to L1 is less than about 0.2 (e.g., between about 0.15 and 0.2, between about 0.1 and 0.2).

In some embodiments, the method optionally includes inserting (before the moving the second elastomeric member) a portion of a bleed fixture into the neck portion 1215 of the container body 1210 via the distal end opening. The bleed fixture is configured to allow the gas G within the dry medicament D to be expelled from the second volume (e.g., the dry medicament volume 1227) via the distal end opening 1218 while limiting the movement of the dry medicament D into the neck portion of the second volume. In this manner, the method can limit the likelihood that a portion of the dry medicament will become compacted near the neck (where the needle is inserted).

In some embodiments, the lyophilized medicament within the container body when the second elastomeric member is at the first distance from the distal end produces a lyophilized cake. The moving of the second elastomeric member to the second distance reduces the size of the lyophilized cake. Moreover, in some embodiments, the resulting lyophilized cake can be broken apart or otherwise have a non-uniform appearance.

After the second elastomeric member is moved, the distal end opening is sealed by installing the distal seal about the distal end opening, at 14. The method then includes conveying the solvent into the first volume via a proximal end opening, at 15. Finally, the first elastomeric member is inserted into the container body via the proximal end opening to seal the solvent within the first volume, at 16.

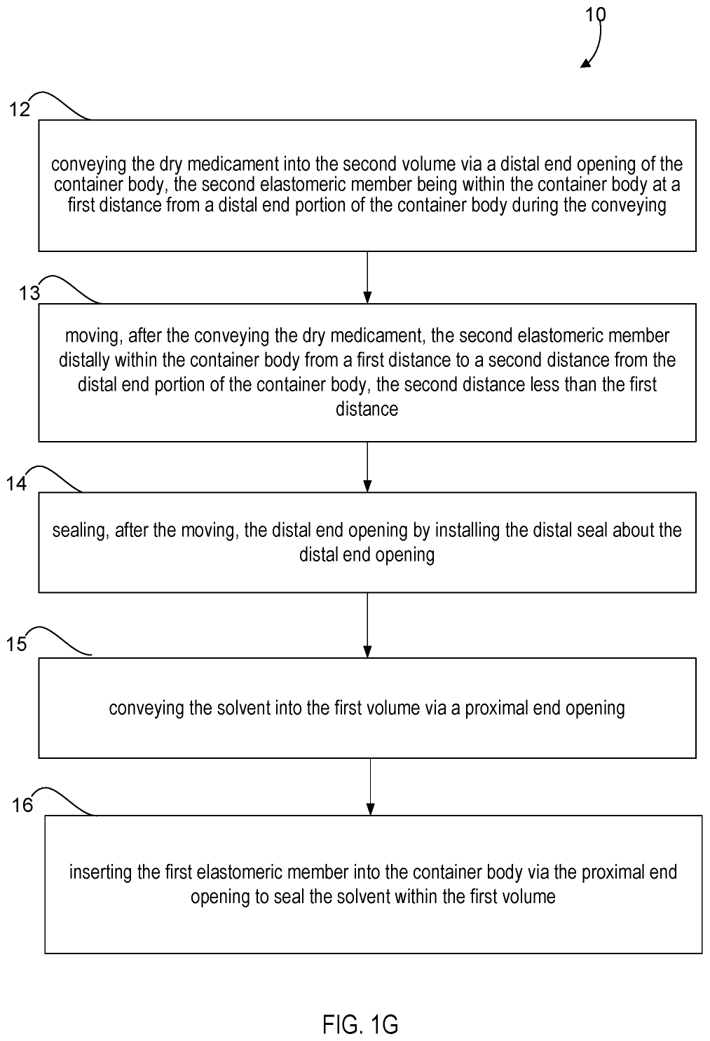

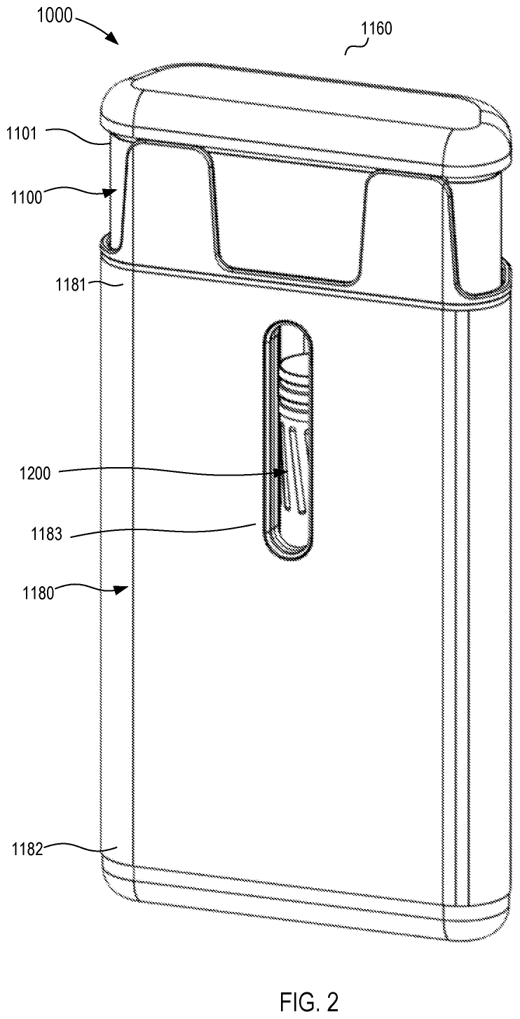

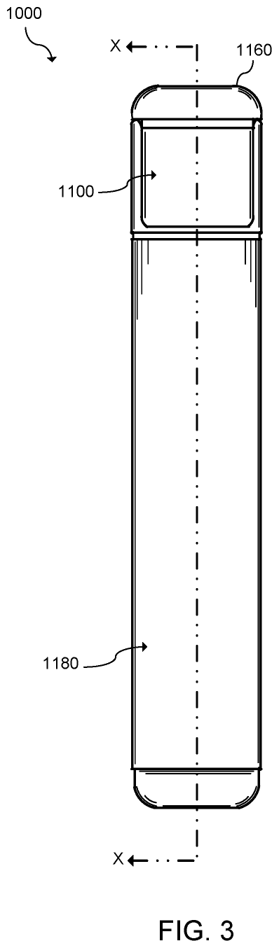

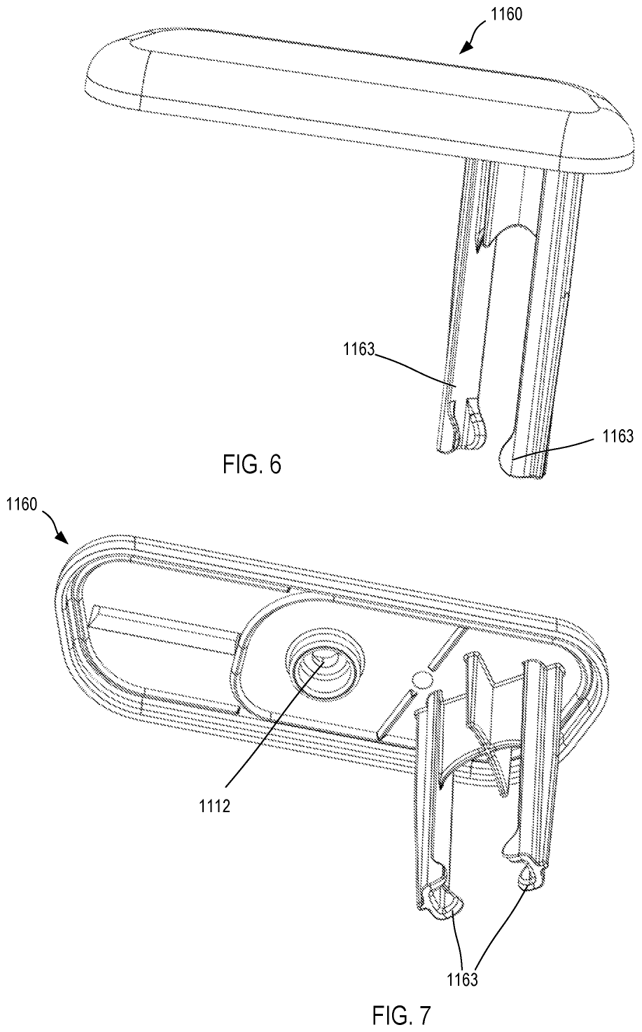

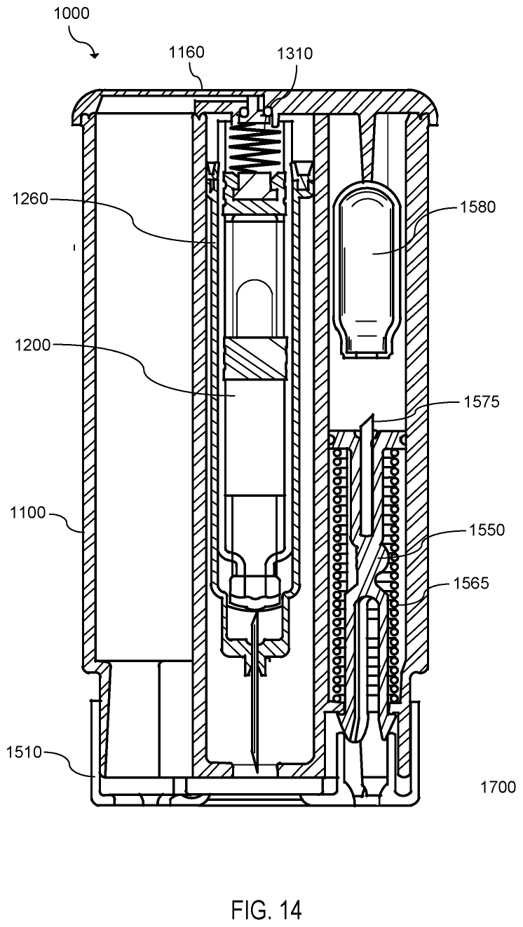

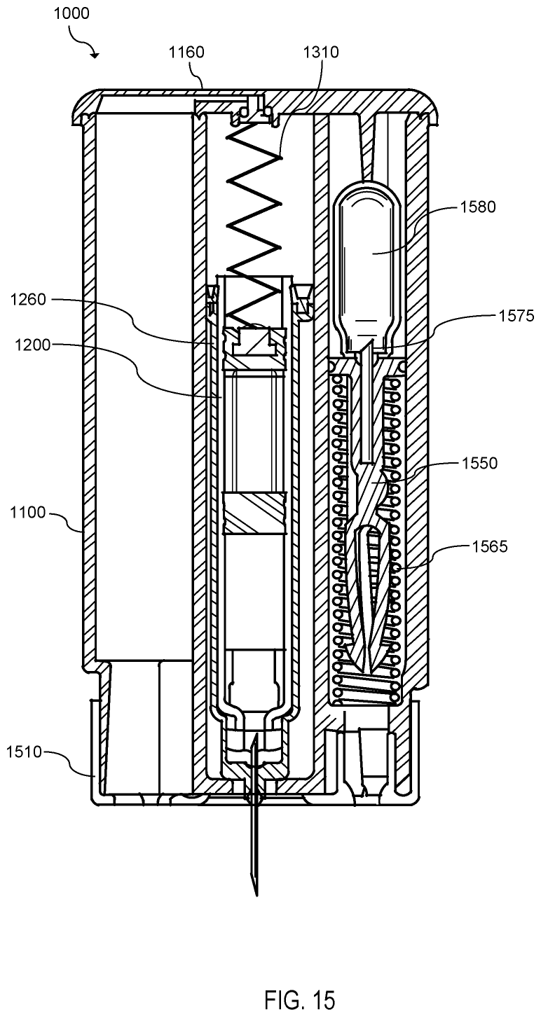

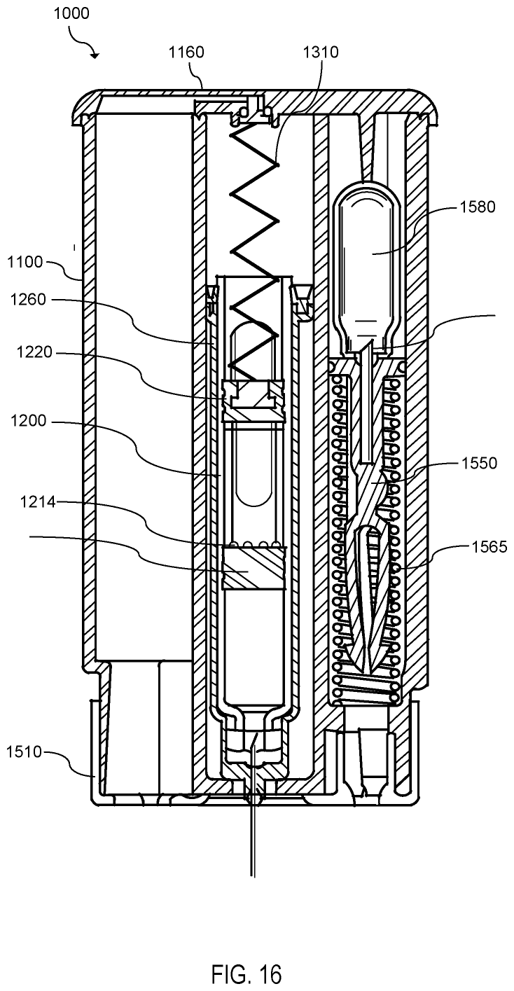

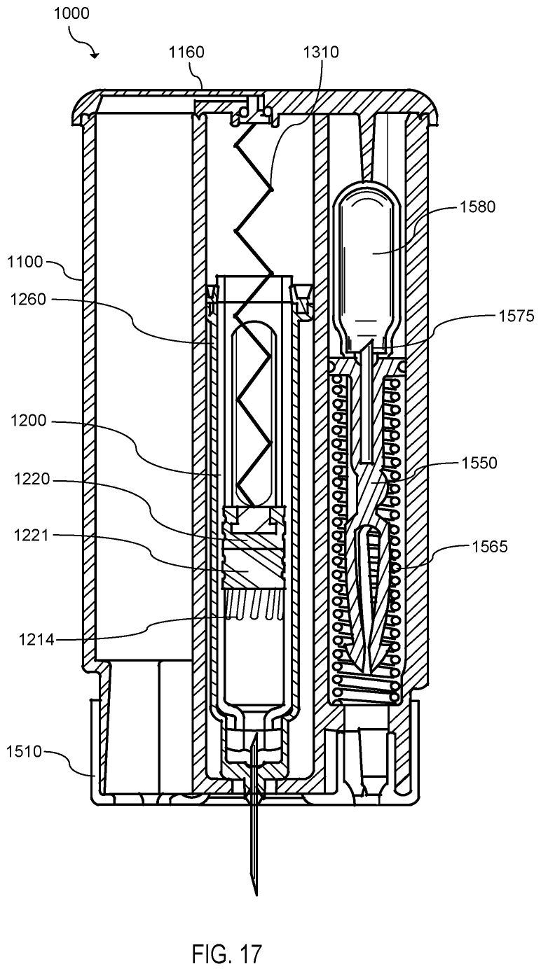

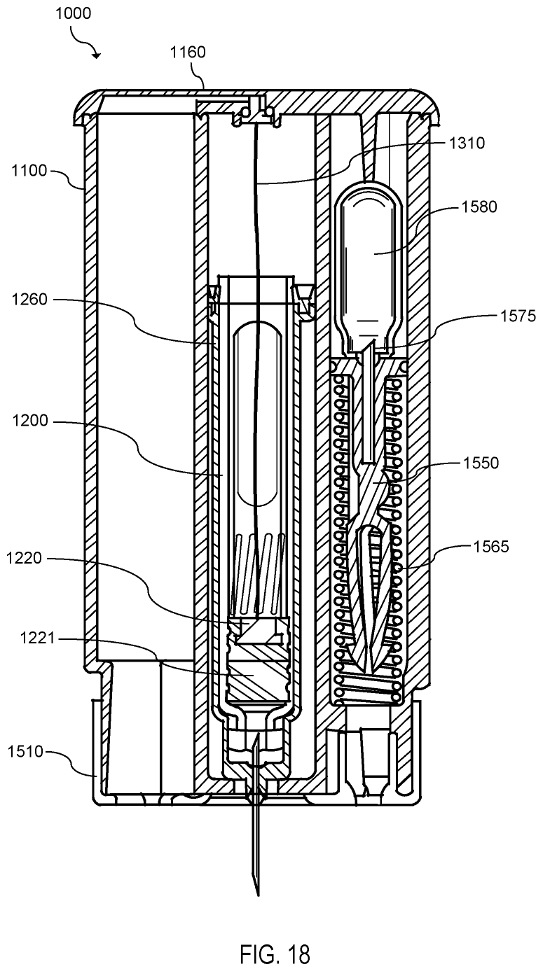

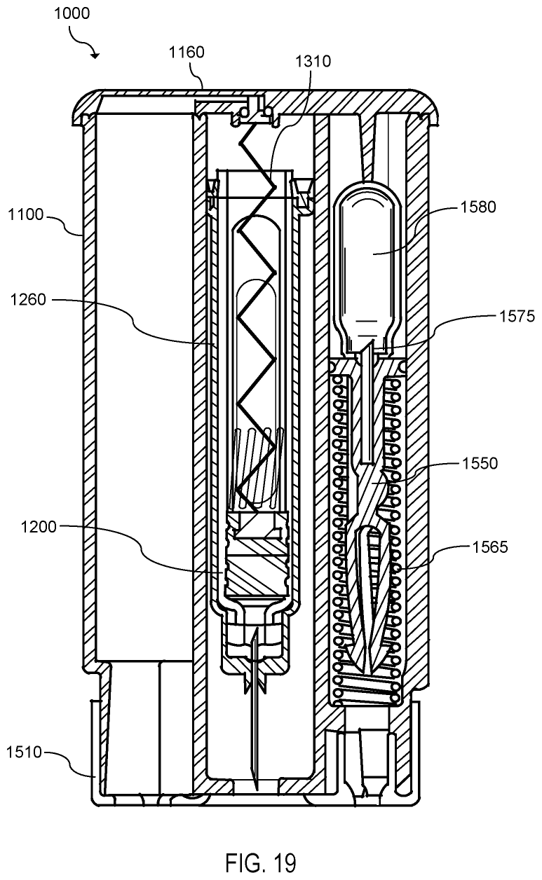

FIGS. 2-19 show various views of a medical injector 1000 according to an embodiment in various different configurations (or stages of operation). FIG. 2 is a perspective view and FIG. 8 is cross-sectional view of the medical injector 1000 (also referred to herein as “medicament delivery device” or “drug product”) in a first configuration (i.e., prior to use). FIG. 14 is a cross-sectional view of the medicament delivery device in a second configuration (with the cover and safety lock removed). FIG. 15 is a cross-sectional view of the medicament delivery device in a third configuration (after being activated). FIG. 16 is a cross-sectional view of the medicament delivery device in a fourth configuration (with the bypass flow being initiated). FIG. 17 is a cross-sectional view of the medicament delivery device in a fifth configuration (with the bypass flow being completed). FIG. 18 is a cross-sectional view of the medicament delivery device in a sixth configuration (with the injection being completed). FIG. 19 is a cross-sectional view of the medicament delivery device in a seventh configuration (with the retraction completed).

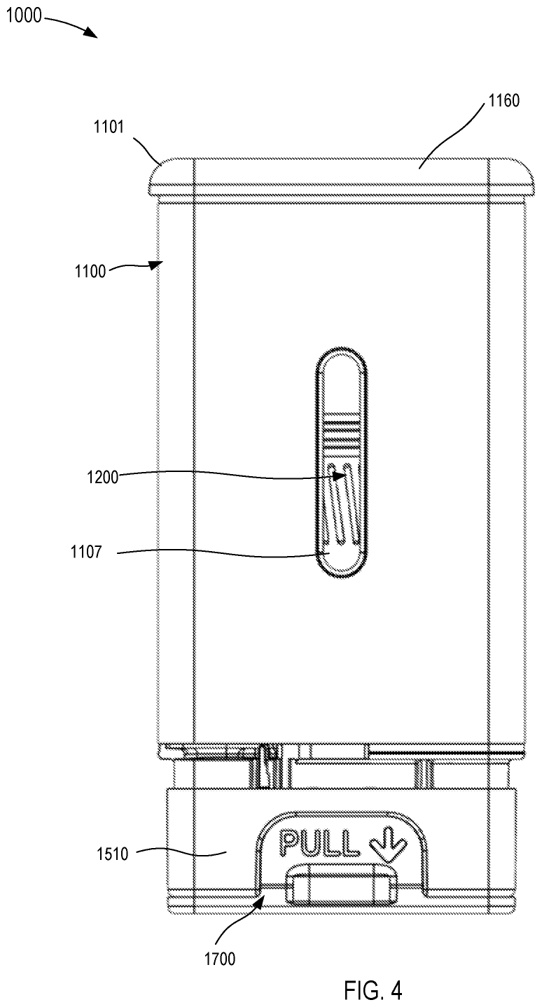

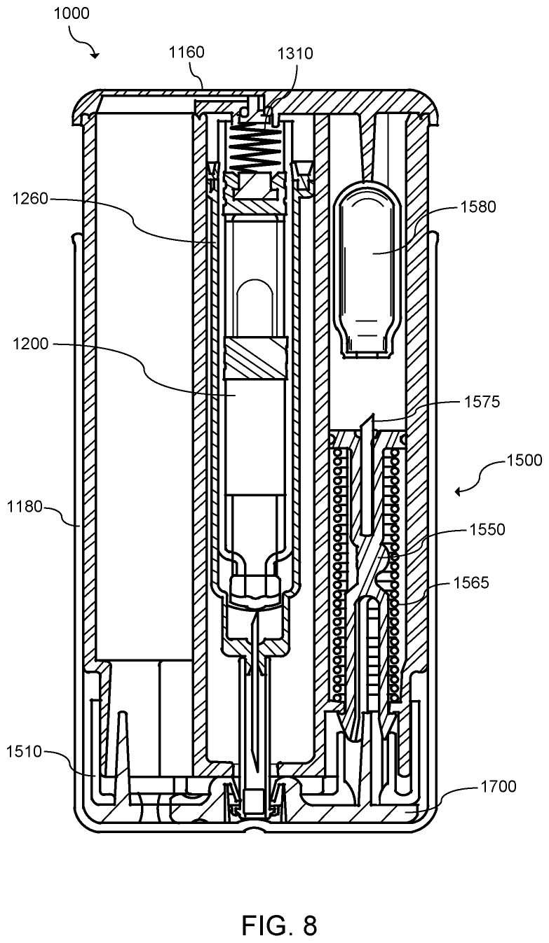

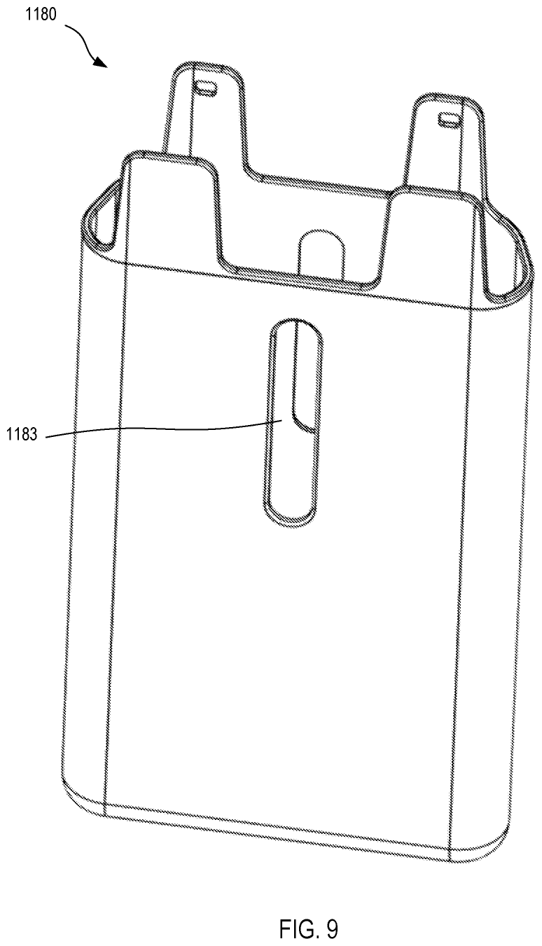

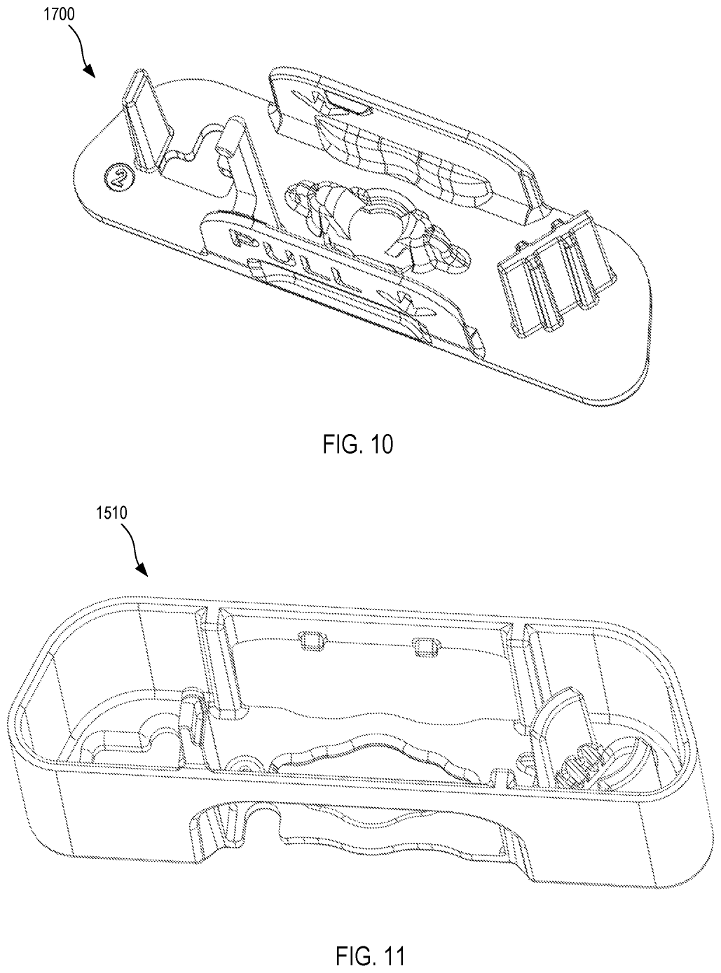

The medical injector 1000 includes a cover 1180 (see FIG. 9), a housing 1100 (see FIG. 5), a system actuation assembly, a medicament container assembly 1200 (see, e.g., FIG. 1F), a base 1510 (or actuator, see FIG. 11); and a safety lock 1700 (see FIG. 10). As shown, the housing 1100 has a proximal end portion 1101 and a distal end portion 1102. The housing 1100 defines a pair of status indicator apertures 1130 disposed on a front side and a rear side of the housing 1100 (e.g., opposite sides of the housing 1100), which are configured to allow a patient to monitor the status and/or contents of the medicament container assembly 1200 contained within the housing 1100. For example, by visually inspecting the status indicator apertures 1130, a patient can determine whether the medicament container assembly 1200 contains a medicament and/or whether the medicament has been dispensed. In some embodiments, the medicament container assembly 1200 can be positioned such that the dry medicament volume 1227 and/or the dry medicament therein are at least partially shielded. In this manner, if the dry medicament is deformed or broken apart by the methods described herein, the user will not improperly interpret such non-uniformities as being indicative of a defective drug product.

As shown in FIG. 5, the inner surface of the housing 1100 defines a gas cavity 1151, a medicament cavity 1139, and a side cavity 1132. The gas cavity 1151 is configured to receive a set of retention members 1163 included in a proximal cap 1160, a gas container 1580, and a portion of the system actuator assembly 1500 (e.g., a release member 1550 and a spring 1565, as shown in FIG. 8). The gas cavity 1151 is at least partially separated from the medicament cavity 1139. The gas cavity 1151 is in fluid communication with the medicament cavity 1139 to allow flow of pressurized gas via a gas passageway (not identified).

The medicament cavity 1139 is configured to receive the medicament container assembly 1200 and the carrier 1260. An inner surface of the housing 1100 includes and/or forms a sidewall that separates the medicament cavity 1139 from the gas cavity 1151, and a sidewall that separates at least a portion of the medicament cavity 1139 from the side cavity 1132. The carrier 1260 and the medicament container assembly 1200 are movable within the medicament cavity 1139 in the proximal direction and in the distal direction. Moreover, the carrier 1260 includes a seal member 1270 configured to form a substantially fluid tight seal with the inner surface of the housing 1100 defining the medicament cavity 1139. The seal member 1270 also seals the carrier 1260 and the container body 1210. Thus the proximal portion of the medicament cavity 1139 forms a gas chamber that is substantially fluidically isolated by the seal member 1270. Specifically, the seal member 1270 has two sealing portions: one that forms a seal against the container body 1210 and the other that forms a seal against the inner surface of the housing 1100. In use, the pressurized gas applies an actuation force in a distal direction against the seal member 1270. The actuation force urges the two sealing portions outward (i.e., one sealing portion outward towards the container body 1210 and the other outward towards the housing 1100), which further enhances sealing. In other embodiments, however, the carrier 1260 can include multiple separate seals (e.g., O-rings) that form a fluid-tight seal with the housing 1100 and the container body 1210.

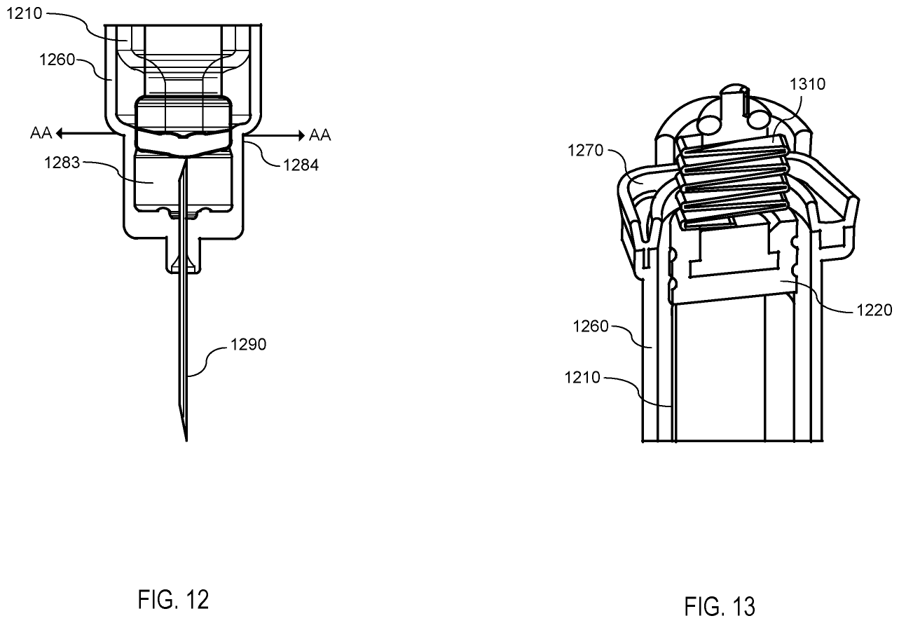

In some embodiments, the proximal portion of the medicament cavity can include a protrusion or stepped feature that limits movement of a retention portion 1284 of the carrier 1260. Referring to FIG. 12, when the medical injector 1000 is in the first configuration, the distal end portion 1212 of the container body 1210 is secured within a retention portion 1284 of the carrier 1260. Thus the distal end portion of the medicament container assembly 1200 is within the coupling volume 1283 but spaced apart from the proximal tip of the needle 1290. As shown in FIG. 15, when the device is actuated the carrier moves distally, and the gas pressure exerted causes the medicament container assembly 1200 to be pushed distally and released from the retention portion 1284 by an outward deformation of the retention portion (see the arrows AA in FIG. 12). The optional proximal safety protrusion can contact the carrier in a manner to prevent inadvertent outward deformation of the retention portion 1284 when the device is in the first configuration. Similarly stated, in some embodiments, the inner wall of the housing 1100 that defines the medicament cavity can include a stepped portion that engages the carrier 1260 near the retention portion 1284. This arrangement limits the likelihood that retention portion 1284 will inadvertently be deformed outward when the device 1000 is in the storage configuration. This additional safety feature can be helpful to prevent the seal of the medicament container assembly 1200 from being punctured by the needle during conditions when the device 1000 may be subjected to high external forces (e.g., vibratory forces, impact forces due to being dropped, etc.).

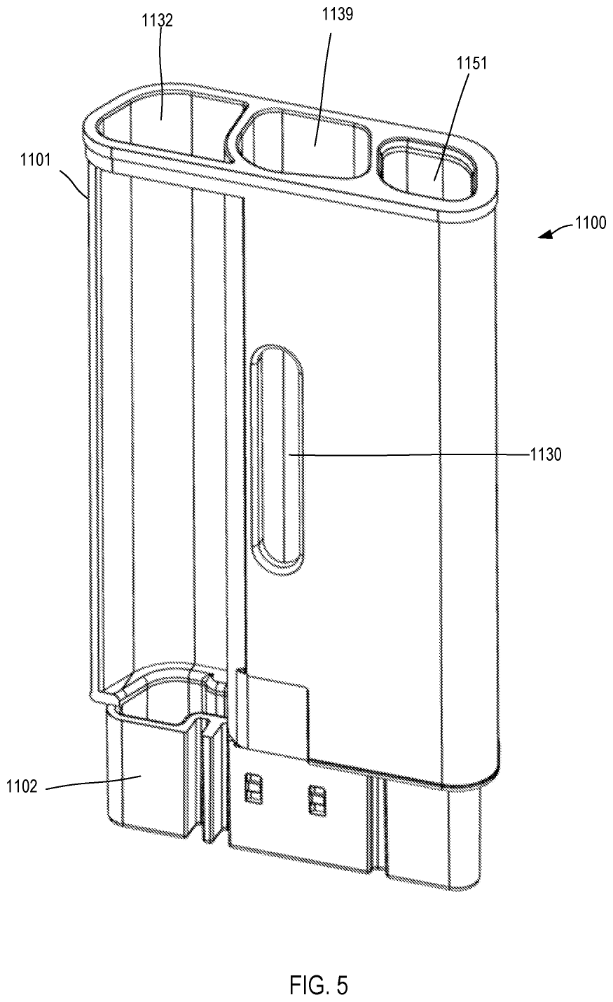

The distal end portion 1102 of the housing 1100 defines a lock rod opening, a needle opening, and a system activation opening. The lock rod opening receives a portion of the safety lock 1700 when the safety lock 1700 is coupled to the housing 1100. The needle opening is the opening through which the needle 1290 is disposed (see e.g., FIGS. 15-18) when the medical injector 1000 is actuated. The system activation opening receives a portion of the rod (e.g., the release member 1550) and allows the system actuator assembly 1500 to be moved in a proximal direction relative to the housing 1100. As described above, the proximal end portion 1101 of the housing 1100 includes and/or is otherwise coupled to a proximal cap 1160 (see e.g., FIGS. 6-7). The proximal cap 1160 includes the retention members 1163. The proximal cap 1160 is coupled to the proximal surface of the housing 1100. In some embodiments, the proximal cap 1103 is fixedly coupled to the proximal surface via, for example, ultrasonic welding, adhesive, fasteners, and/or the like or a combination thereof. Moreover, a seal member can be disposed in a seal recess to form a substantially fluid tight seal between the proximal cap 1160 and the proximal surface of the housing 1100.

The retention members 1163 of the proximal cap 1160 are configured to receive and/or retain the gas container 1580 that contains a pressurized gas. When the medical injector 1000 is actuated, pressurized gas from the gas container 1580 is conveyed from the gas cavity 1151 to the medicament cavity 1139 via the gas passageway.

FIG. 9 shows the cover 1180 of the medical injector 1000. The cover 1180 can be any suitable configuration and can include any suitable feature to house, contain and/or protect portions of the medical injector 1000. The cover 1180 includes a proximal end portion 1181 and a distal end portion 1182, and defines a cavity and a set of status windows 1183. The cavity of the cover 1180 is configured to receive at least a portion of the housing 1100. The status windows 1183 are disposed on opposite sides of the cover 1180 and are configured such that, when the portion of the housing 1100 is disposed within the cover 1180, the status windows 1183 of the cover 1180 are at least partially aligned with the corresponding status indicator aperture 1130 of the housing 1100. Thus, a user can visually inspect a portion of the medicament container assembly 1200 via the status windows 1183 of the cover 1180 and the status indicator apertures 1130 of the housing 1100. The cover 1180 also functions as a safety mechanism by preventing movement of the safety lock 1700 (e.g., a safety guard) and by preventing inward movement of the base 1510.

FIG. 10 shows the safety lock 1700 of the medical injector. The safety lock has similar structure and function as the safety locks shown in International Patent Publication No. WO2017/004345, entitled “Auto-Injectors for Administration of a Medicament Within a Prefilled Syringe,” filed Jun. 30, 2016 (“the '4345 PCT”) and International Patent Publication No. WO2020/140040, entitled “Devices and Methods for Delivery of Substances Within a Prefilled Syringe,” filed Dec. 27, 2019 (“the '0040 PCT”), each of which is incorporated herein by reference in its entirety.

FIG. 11 shows the base 1510 (which functions as an actuator) of the medical injector. The base has similar structure and function as the safety locks shown in the '4345 PCT and the '0040 PCT, each of which is incorporated herein by reference in its entirety.

FIG. 13 shows the gas release assembly 1310 of the medical injector. The gas release assembly 1310 expands as the first elastomeric member 1220 moves distally and serves to release a gas vent when the end of the injector stroke is reached. The gas release assembly 1310 a has similar structure and function as the gas release (or expandable) assemblies shown in the '4345 PCT and the '0040 PCT, each of which is incorporated herein by reference in its entirety. In some embodiments the side cavity 1132 of the housing can receive the vented gas. In some embodiments, the side cavity 1132 can include an acoustic structure to produce an audible indicator (e.g., a whistling sound) when the gas is released.

FIGS. 14-19 show the medical injector in its various states of delivery.

After the cover 1180 is removed from the housing 1100, the medical injector 1000, the safety lock 1700 is exposed and can be removed. FIG. 14 shows the medical injector in the second configuration with the cover 1180 and the safety lock 1700 both removed.

Referring to FIG. 15, the medical injector 1000 can then be activated by moving the base 1510 from a first position to a second position to place the medical injector 1000 in the third configuration. Similarly stated, the medical injector 1000 can be activated by the system actuator assembly 1500 by moving the base 1510 proximally relative to the housing 1100. The base 1510 is moved from its first position to its second position by placing the medical injector 1000 against a target surface (e.g., the body of the patient) and moving the base 1510 with respect to the housing 1100 in the proximal direction.

The proximal movement of the base 1510 from its first position to its second position actuates and/or otherwise releases the release member 1550. As such, the spring 1565 is allowed to transition from a first configuration (e.g., a compressed configuration) to a second configuration (e.g., a non-compressed configuration), thereby moving the release member 1550 within the gas cavity 1151. Proximal movement of the release member 1550 causes the puncturer 1575 to puncture and/or pierce a portion of the gas container 1580 (e.g., a frangible seal or the like). After the gas container 1580 has been punctured, an actuating portion of a compressed gas flows from the gas container 1580 and into the gas cavity 1151. Moreover, with a seal of the release member 1550 forming a substantially fluid tight seal with the inner surface defining the gas cavity 1151, the actuating portion of the compressed gas fills the gas cavity 1151 and is forced through the gas passageway and into the medicament cavity 1139.

As the gas flows into the medicament cavity 1141, the gas applies gas pressure to the upper portion of the carrier 1260 (including the carrier seal 1270) and the first elastomeric member 1220 within the medicament container 1210. This gas force (the insertion force) causes the medicament container 1210, the carrier 1240 and the needle 1290 to contemporaneously move within the housing 1100 in the distal direction. The movement of the needle 1290 in a distal direction causes the distal end portion of the needle 1290 to exit the housing 1100 and enter the body of a patient prior to administering the medicament.

Referring to FIG. 16, after the needle 1290 is inserted the continued build-up of gas pressure moves the first elastomeric member 1220 and the second elastomeric member 1221 distally to a position where the bypass 1214 is aligned with the second elastomeric member 1221. In this embodiment, the bypass 1214 can extend along a length of the medicament container 1210 that is greater that a length along which each elastomeric member 1220 and 1221 extends. Thus, when the bypass 1214 is substantially aligned with the second elastomeric member 1221, the increased pressure within the solvent volume 1226 urges a flow of the liquid solvent through the bypass 1214 and around the second elastomeric member 1221 to be transferred into the dry medicament volume 1227. In this manner, the solvent can mix with the dry medicament disposed within the dry medicament volume 1227 to reconstitute the medicament for injection. As described above, the bypass channels 1214 and the pressure exerted by the movement of the elastomeric members can produce sufficient fluidic mixing within the dry medicament volume 1227 to eliminate the need for shaking, inverting the injector, or any other steps. FIG. 17 shows the medical injector at the end of the movement the elastomeric members (just prior to injection of the reconstituted drug).

Additionally, as described herein, the delivery sequence is devoid of any separate priming operation (also referred to as a gas venting operation). The dry medicament is formulated to be reconstituted quickly, and the limited amount of gas present within the medicament container assembly 1200 obviates the need for additional time (for dissolution) or gas venting.

As shown in FIGS. 17 and 18, the first elastomeric member 1220 can be in contact with the second elastomeric member 1221 such that the gas pressure exerts a force sufficient to move both the elastomeric members 1220 and 1221 in the distal direction. The distal movement of the elastomeric members 1220 and 1221 generates a pressure upon the medicament contained within the container body 1210, thereby allowing at least a portion of the medicament to flow out of the container body 1210 via the needle 1290, as shown in FIG. 18. Furthermore, when the elastomeric members 1220 and 1221 are disposed in a distal position within the medicament container 1210, the medical injector 1000 has delivered a dose of the reconstituted medicament.

FIG. 19 shows the medical injector in its retracted position. The retraction of the carrier 1260 and the needle is occurs in response to the pressurized gas within the gas chamber of the housing being vented by the expansion of the gas release assembly 1310. Specifically, when the first elastomeric member 1220 reaches the desired position (i.e., its distal-most position corresponding to the end of injection), the gas release assembly 1310 actuates a valve to release the gas. Operation of the gas release assembly 1310 can be similar to that shown in the '4345 PCT and the '0040 PCT, each of which is incorporated herein by reference in its entirety. After the gas pressure within the gas chamber is reduced, a retraction spring (not shown) urges the carrier 1260 proximally.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments as discussed above.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

Although the medicament delivery devices are shown and described herein as being medical injectors having a medicament container divided into two portions (see e.g., the medical injector 1000), in other embodiments, any of the components, methods and/or formulations described herein can be used in any suitable medicament delivery device. In some embodiments, the medicament delivery device can include a medicament container having any number of plungers and/or defining any number of volumes therein.

Although the medicament container 1210 is shown as being initially spaced apart from and/or fluidically isolated from the needle 1290, in other embodiments, a medical injector 1000 can include a medicament container that has a staked needle. For example, in some embodiments, a medical injector 1000 includes a prefilled syringe in which the needle is in fluid communication with the medicament container. In such embodiments, the mixing operation need not, therefore, place the container in fluid communication with the needle.

Although the components and methods described herein are shown and described as being included in devices that include a medicament, in other embodiments, any of the components and/or methods described herein can be used in either an actual medicament delivery device or a simulated medicament delivery device. A simulated medicament delivery device, for example, can correspond to an actual medicament delivery device and can facilitate the training of a user in the operation of the corresponding actual medicament delivery device. A simulated medicament delivery device or trainer can be similar to the simulated medicament delivery devices or trainers described in U.S. Patent Publication Number 2008/0059133, entitled “Medical Injector Simulation Device,” filed Feb. 27, 2007, which is incorporated herein by reference in its entirety.

In such embodiments, the simulated medicament delivery device can simulate the actual medicament delivery device in any number of ways. For example, in some embodiments, the simulated medicament delivery device can have a shape corresponding to a shape of the actual medicament delivery device, a size corresponding to a size of the actual medicament delivery device and/or a weight corresponding to a weight of the actual medicament delivery device. Moreover, in some embodiments, the simulated medicament delivery device can include components that correspond to the components of the actual medicament delivery device. In this manner, the simulated medicament delivery device can simulate the look, feel, forces, and sounds of the actual medicament delivery device. For example, in some embodiments, the simulated medicament delivery device can include external components (e.g., a housing, a needle guard, a sterile cover, a safety lock or the like) that correspond to external components of the actual medicament delivery device. In some embodiments, the simulated medicament delivery device can include internal components (e.g., an actuation mechanism, a compressed gas source, a medicament container or the like) that correspond to internal components of the actual medicament delivery device.

In some embodiments, however, the simulated medicament delivery device can be devoid of a medicament and/or those components that cause the medicament to be delivered (e.g., a needle, a nozzle or the like). In this manner, the simulated medicament delivery device can be used to train a user in the use of the actual medicament delivery device without exposing the user to a needle and/or a medicament. Moreover, the simulated medicament delivery device can have features to identify it as a training device to prevent a user from mistakenly believing that the simulated medicament delivery device can be used to deliver a medicament. For example, in some embodiments, the simulated medicament delivery device can be of a different color than a corresponding actual medicament delivery device. Similarly, in some embodiments, the simulated medicament delivery device can include a label and/or other indicia clearly identifying it as a training device.

Any of the medicament containers described herein can be any container suitable for storing the compositions disclosed herein. In some embodiments, the medicament container can be a pre-filled syringe, a pre-filled cartridge, a vial, or the like. In some embodiments, for example, any of the devices shown and described herein can include components and/or mechanisms to accommodate a pre-filled syringe, similar to the embodiments shown and described in U.S. Patent Publication No. 2013/0023825 entitled, “Medicament Delivery Devices for Administration of Medicament within a Prefilled Syringe,” filed Jan. 25, 2012 the disclosure of which is incorporated herein by reference in its entirety. In other embodiments, the medicament containers described here can be a container having a flexible wall, such as, for example, a bladder.

Any of the devices and/or medicament containers shown and described herein can be included in a kit (not shown), which can include fungible components and reusable components. For example, in some embodiments, at least a housing of a medical injector can be reusable without the need for sterilization, as described in detail above. In some embodiments, such as with the medical injector 1000, the proximal cap 1160 can be removed from the housing 1100 to allow access to and removal of the used components disposed within the housing 1100. In addition, the removal of the proximal cap 1160 from the housing 1100 can allow for any suitable portion of the medical injector 1000 to be reset to, for example, a pre-activated or pre-actuated configuration, as described above.

### Medicaments and Formulations

Any of the devices and/or medicament containers shown and described herein can include any suitable medicament or therapeutic agent. For example, although the medical injectors described above are shown and described as including a multi-chamber medicament container (e.g., medicament container 1210) that includes a substantially dry medicament (e.g., contained within the dry medicament volume 1227) and a solvent (e.g., contained within the solvent volume 1226), in other embodiments, any of the medicament delivery devices disclosed herein can include a multi-chamber container that is filled with any suitable substances. For example, in some embodiments, any of the medicament delivery devices disclosed herein can include a medicament container (e.g., a cartridge) that separately stores and mixes, upon actuation, two liquid substances. For example in some embodiments, any of the devices shown and described herein can include a medicament container filled with (in separate chambers) epinephrine and at least one antihistamine (e.g., epinephrine and diphenhydramine, epinephrine and hydroxyzine, epinephrine and cetirizine); an antipsychotic medicament and a benzodiazepine (e.g. haloperidol and diazepam, haloperidol and midazolam, haloperidol and lorazepam); insulin and a GLP-1 analog or incretin mimetic (e.g. insulin and exenatide, insulin and lixisenatide); an NSAID and an opiode (e.g., ketorolac and buprenorphine). Other suitable compositions that can be included in any of the medicament containers and/or devices described herein include pralidoxime chloride and atropine; obidoxime chloride and atropine; epinephrine and atropine; methotrexate and etanercept; methotrexate and adalimumab; and methotrexate and certolizumab.

In some embodiments, a dry medicament composition can include a dose of atropine base of between about 1.5 mg and about 1.7 mg of atropine base. In other embodiments, a dry medicament composition can include a dose of atropine base of between about 1.9 mg and about 2.1 mg of atropine sulfate. In such embodiments, the drug product can include a sufficient amount of solvent to produce a single dose of about 2 mL of drug solution.

In some embodiments, a composition can include glucagon and/or any pharmaceutically acceptable constituents for use in the medicament delivery devices disclosed herein. In some embodiments, the glucagon formulation can be prepared and/or filled according to any suitable method such as, for example, those described in U.S. Patent Publication No. 2013/0023822 incorporated by reference hereinabove. A composition according to an embodiment can be formulated such that the target concentration of glucagon in the solution, either before lyophilization and/or after being reconstituted upon actuation of the device, is approximately 1 mg/mL. In other embodiments, the target concentration of glucagon in the solution, either before lyophilization and/or after being reconstituted, can be approximately 2 mg/mL, approximately 1.5 mg/mL, approximately 0.5 mg/mL (e.g., a pediatric dose) or approximately 0.25 mg/mL. In other embodiments, a composition can be formulated such that the target concentration of glucagon in the solution, either before lyophilization and/or after being reconstituted upon actuation of the device, is between approximately 0.25 mg/mL and 2 mg/mL, between approximately 0.5 mg/mL and 1 mg/mL, or between approximately 0.8 mg/mL and 1.2 mg/mL.

In certain embodiments, the concentration (either before lyophilization or upon reconstitution) of glucagon in a glucagon formulation is about 1 mg/mL and the total solute concentration is about 50 mg/mL. For example, in some embodiments, a composition can include glucagon and any suitable bulking agents to increase the total solute concentration in the glucagon formulation. In this manner, the glucagon formulation can be more effectively lyophilized and/or reconstituted. For example, in some embodiments, as described below, certain bulking agents can be used to improve the stability, solubility and/or efficacy of the composition when reconstituted in any of the devices shown and described herein. In some embodiments, certain bulking agents can be used to produce a visual indicia when the composition is reconstituted (e.g., such agents can allow the reconstituted medicament to be more easily detected by the user).

In some embodiments, a composition can include a peptide, such as, for example, glucagon and a carbohydrate. In this manner, the stability of the peptide (e.g., glucagon) can be increased during lyophilization and subsequent storage. In particular, the stability of peptides, such as glucagon, can be increased in an amorphous (i.e. non-crystalline) environment. It is believed that carbohydrates undergoing dehydration create a solid-state environment that is amorphous and exhibits high viscosity when maintained below the glass transition temperature. In addition, carbohydrates contain multiple hydroxyl groups that may form hydrogen bonds with polar groups on a protein or peptide surface in an amorphous solid-state environment. Without being bound by any particular mechanism, when water is removed during lyophilization, such carbohydrates may maintain the hydrogen bonds and preserve the native-like solid state of the polypeptide structure. In certain embodiments, therefore, the glucagon formulations include other excipients, such as, but not limited to carbohydrates. Suitable carbohydrates include, but are not limited to, lactose, trehalose, mannitol, and combinations thereof.

Additionally, the solubility of glucagon increases below a pH of 4. In certain embodiments, the glucagon formulations, prior to lyophilization and/or after reconstitution, have a pH of less than about pH 5.0, including less than about pH 4.5, less than about pH 4.0, less than about pH 3.5, less than about pH 3.0, less than about pH 2.5, less than about pH 2.0. In other embodiments of the invention, the glucagon formulations, prior to lyophilization and/or after reconstitution, have a pH range of about pH 1.5 to about pH 5.0, inclusive of all ranges and subranges therebetween, e.g., about pH 2.0 to about pH 4.5, about pH 2.0 to about pH 4.0, about pH 2.0 to about pH 3.5, about pH 2.0 to about pH 3.0, about pH 2.0 to about pH 2.5, about pH 2.5 to about pH 4.5, about pH 2.5 to about pH 4.0, about pH 2.5 to about pH 3.5, about pH 2.5 to about pH 3.0, about pH 3.0 to about pH 4.5, about pH 3.0 to about pH 4.0, about pH 3.0 to about pH 3.5, about pH 3.5 to about pH 4.5, and about pH 3.5 to about pH 4.0. In certain embodiments, the pH of the glucagon formulation is adjusted prior to lyophilization by the addition of a suitable acid, such as hydrochloric acid or citric acid.

The lyophilized or dry formulations of the present invention may be reconstituted by any suitable solvent or combination of solvent, including, but not limited to, water, sterile water, glycerin, or hydrochloric acid.

As described above, in some embodiments, a dry formulation can include any suitable bulking agents and/or excipients.

In some embodiments, the medicament contained within any of the medicament containers shown herein can be a vaccine, such as, for example, an influenza A vaccine, an influenza B vaccine, an influenza A (H1N1) vaccine, a hepatitis A vaccine, a hepatitis B vaccine, a haemophilus influenza Type B (HiB) vaccine, a measles vaccine, a mumps vaccine, a rubella vaccine, a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine and/or a meningococcus vaccine. In other embodiments, the medicament contained within any of the medicament containers shown herein can be epinephrine. In other embodiments, the medicament contained within any of the medicament containers shown herein can be naloxone, including any of the naloxone formulations described in U.S. patent application Ser. No. 13/036,720, entitled “Medicament Delivery Device for Administration of Opioid Antagonists Including Formulation for Naloxone,” filed on Feb. 28, 2011, the disclosure of which is incorporated herein by reference in its entirety.

In other embodiments, the medicament contained within any of the medicament containers shown herein can include insulin, glucagon, human growth hormone (HGH), erythropoiesis-stimulating agents (ESA), DeMab, Interferon and other chronic therapies, or the like. Such formulations can be produced using a general lyophilization process with glucagon (of recombinant origin) using bulking agents, stabilizers, buffers, acidifying agents or other excipients comprising of, but not limited to, one or more of the following combinations: lactose, hydrochloric acid; glucose, histidine, hydrochloric acid; trehalose, mannitol, citrate; trehalose, mannitol, hydrochloric acid; trehalose, glycine, hydrochloric acid; Mannitol, ascorbic acid; and Glycine, hydrochloric acid.

In other embodiments any of the injectors described herein can be filled with and/or used to inject medicament formulations, including lyophilized biologics and/or biopharmaceuticals, such as, for example, canakinumab, certolizumab, golimumab, and/or interleukins, for the treatment of crypyrin associated periodic syndromes, hereditary andioedema, and other auto-immune diseases. In yet other embodiments any of the injectors described herein can be filled with and/or used to inject intranasal biologics, such as glucagon or human growth hormone, formulated for use in an auto injector, for the treatment of musculoskeletal diseases, growth disorders, diabetes & treatment related disorders.

In other embodiments, any of the injectors described herein can be filled with and/or used to inject an anti-thrombotics, such as LMWH, ULMWH, Xa Inhibitors, biotinylated idraparinux, etc., for either the acute management and/or surgical prophylaxis of deep vein thrombosis and/or pulmonary embolism or for the management of other conditions which may require anticoagulation to prevent thromboembolism, such as its use in cardiovascular diseases including atrial fibrillation and ischemic stroke. In another example, in some embodiments an injector according to an embodiment can be filled with and/or used to inject formulations for the treatment of asthma and/or chronic obstructive pulmonary disease.

In other embodiments, any of the injectors described herein can be filled with and/or used to inject recombinant hyaluronidase.

In other embodiments, any of the injectors described herein can be filled with and/or used to inject depot medroxyprogesterone acetate for the treatment of infertility.

In other embodiments, any of the injectors described herein can be filled with and/or used to inject environmental, food, and household allergen formulations for the treatment of allergic disease, specifically for use in immunotherapy.

In still other embodiments, the medicament contained within any of the medicament containers shown herein can be a placebo substance (i.e., a substance with no active ingredients), such as water.

The medicament containers and/or medicament delivery devices disclosed herein can contain any suitable amount of any medicament. For example, in some embodiments, a medicament delivery device as shown herein can be a single-dose device containing an amount of medicament to be delivered of approximately 0.4 mg, 0.8 mg, 1 mg, 1.6 mg or 2 mg. As described above, the fill volume can be such that the ratio of the delivery volume to the fill volume is any suitable value (e.g., 0.4, 0.6 or the like).

In some embodiments, a method includes moving a first elastomeric member within a medicament container such that a medicament within a first chamber is compressed. The medicament can be, for example, a substantially solid medicament, such as a lyophilized medicament that that contains air therein. In other embodiments, the medicament within the first chamber can include a liquid component, and the first chamber can include air. In this manner, a portion of the air within the first chamber can be conveyed (or purged) from the first chamber. As described herein, in some embodiments, the air from the first chamber can be conveyed into a second chamber of the medicament container. In some embodiments, the method includes puncturing a second elastomeric member or seal member, which defines a boundary of the second chamber such that a portion of the air within the second chamber is conveyed via the needle to volume outside of the medicament container. In other embodiments, the air from the first chamber can be conveyed to a volume outside of the medicament container.

What is claimed is:

1. A method of producing a medicament container assembly that contains a dry medicament and a solvent, the medicament container assembly including a container body, a first elastomeric member, a second elastomeric member, and a distal seal, the first elastomeric member disposed within a proximal end portion of the container body, the second elastomeric member disposed within the container body distally from the first elastomeric member, the first elastomeric member, the second elastomeric member, and a portion of the container body collectively defining a first volume, the second elastomeric member and a distal end portion of the container body defining a second volume, the method comprising:
conveying the dry medicament into the second volume via a distal end opening of the container body, the second elastomeric member being within the container body at a first distance from a distal end portion of the container body during the conveying;
moving, after the conveying the dry medicament, the second elastomeric member distally within the container body to a second distance from the distal end portion of the container body, the second distance being less than the first distance;
sealing, after the moving, the distal end opening by installing the distal seal about the distal end opening;
conveying the solvent into the first volume via a proximal end opening; and
inserting the first elastomeric member into the container body via the proximal end opening to seal the solvent within the first volume.

2. The method of claim 1, wherein:
the dry medicament is a lyophilized medicament; and
the conveying the dry medicament includes:
conveying a liquid medicament into the second volume, and
lyophilizing the liquid medicament to produce the lyophilized medicament.

3. The method of claim 2, wherein:
the lyophilized medicament within the container body at a first distance from the distal end produces a lyophilized cake; and
the moving the second elastomeric member distally within the container body to the second distance reduces the size of the lyophilized cake.

4. The method of claim 1, wherein the conveying the dry medicament into the second volume is performed via a dry fill process.

5. The method of claim 4, wherein the dry medicament is produced by any of a spray drying process or a micronizing process.

6. The method of claim 1, wherein:
the moving the second elastomeric member causes a gas within the dry medicament to be expelled from the second volume via the distal end opening.

7. The method claim 1, wherein:
the moving the second elastomeric member causes a size of the second volume to be decreased by at least fifty percent.

8. The method of claim 1, wherein:
the moving the second elastomeric member causes a size of the second volume to be less than 1 mL.

9. The method of claim 1, wherein the distal end portion of the container body includes a neck and the second volume includes a neck portion, the method further comprising:
inserting, after the conveying the dry medicament and before the moving the second elastomeric member, a portion of a bleed fixture into the neck portion of the second volume via the distal end opening, the bleed fixture configured to allow a gas within the dry medicament to be expelled from the second volume via the distal end opening while limiting the movement of the dry medicament into the neck portion of the second volume.

10. The method of claim 1, wherein:
the container body includes a bypass channel configured to fluidically couple the first volume and the second volume when the second elastomeric member is at least partially aligned with the bypass channel; and
the second elastomeric member is located proximally from the bypass channel when the second elastomeric member is within the container body at the first distance and the second distance.

11. The method of claim 10, wherein:
a proximal end portion of the bypass channel is at a third distance from the distal end portion of the container body, a ratio of the third distance to a length of the container body being less than 0.20.

12. The method of claim 1, wherein the solvent is aqueous.

13. The method of claim 12, wherein the aqueous solvent includes an alcohol or miscible organic solvent.

14. The method of claim 12, wherein the aqueous solution includes a surfactant.

15. An apparatus, comprising:
a medicament container produced according to the method of claim 1.

16. A method of producing a medicament container assembly that contains a dry medicament and a solvent, the medicament container assembly including a container body, a first elastomeric member, a second elastomeric member, and a distal seal, the first elastomeric member disposed within a proximal end portion of the container body, the second elastomeric member disposed within the container body distally from the first elastomeric member, the first elastomeric member, the second elastomeric member, and a portion of the container body collectively defining a first volume, the second elastomeric member and a distal end portion of the container body defining a second volume, the method comprising:
dry filling the dry medicament into the second volume via a distal end opening of the container body, the second elastomeric member being within the container body at a first distance from a distal end portion of the container body during the conveying;
sealing, after the moving, the distal end opening by installing the distal seal about the distal end opening;
conveying the solvent into the first volume via a proximal end opening; and
inserting the first elastomeric member into the container body via the proximal end opening to seal the solvent within the first volume.

17. The method of claim 16, wherein the dry medicament is produced by any of a spray drying process or a micronizing process.

18. An apparatus, comprising:
a housing having an inner wall that defines a medicament cavity;
a medicament container assembly at least partially disposed within the medicament cavity of the housing, the medicament container assembly containing a dose of a medicament; and
a carrier configured to move within the medicament cavity from a first carrier position to a second carrier position in response to an actuation force, the carrier including a side wall that includes a retention portion and defines a coupling volume, the carrier coupled to a needle having a proximal tip that extends into the coupling volume, the retention portion of the carrier configured to retain a distal end portion of the medicament container assembly in a first container position, the distal end portion of the medicament container assembly fluidically isolated from the proximal tip of the needle when the medicament container assembly is in the first container position, the distal end portion of the medicament container assembly configured to move to a second container position in response to the actuation force, the distal end portion of the medicament container assembly being fluidically coupled to the proximal tip of the needle when the medicament container assembly is in the second container position,
wherein the inner wall of the housing and the side wall of the carrier are configured to limit deformation of the retention portion to prevent the medicament container assembly from moving to the second container position when the carrier is in the first carrier position.
